(19) United States
(12) Patent Application Publication
(10) Pub. No.: US 2024/0004105 A1
(43) Pub. Date: Jan. 4, 2024

(54) REFRACTIVE-INDEX ADUSTABLE LENS

(71) Applicant: Griffith ALTMANN, Ladera Ranch, CA (US)

(72) Inventor: Griffith ALTMANN, Ladera Ranch, CA (US)

(21) Appl. No.: 18/027,549

(22) PCT Filed: Sep. 23, 2020
(86) PCT No.: PCT/IB2021/058614

§ 371 Date: Mar. 21, 2023

Related U.S. Application Data
(60) Provisional application No. 63/082,336, filed on Sep. 23, 2020.

(51) Int. Cl. G02B 1/12; G02B 3/00

(52) U.S. Cl. CPC G02B 1/12; G02B 3/0062

(57) ABSTRACT

A lens comprising an optic body may include a first portion comprised of a first material having a first spectral transmission profile and a second portion comprised of a second material having a second spectral transmission profile. A beam of light may be transmitted through the first portion to be absorbed by the second portion to effect a refractive-index change in the second material.

### FIELD

The subject matter disclosed herein concerns a lens that may be irradiated to change the refractive index of the lens.

### BACKGROUND

Prescription lenses for improving vision include spectacle lenses, contact lenses, and intraocular lenses. When a subject's vision changes, the subject typically obtains new lenses having different optical properties that account for the vision change.

Refractive-index adjustable lens materials and laser-based techniques for modifying their refractive indices have been described. See, e.g., U.S. Pat. No. 9,060,847 and U.S. Patent Application Publication No. 2006/0135952. The laser-based techniques may be based on one-photon absorption (1PA”) or two-photon absorption (“2PA”). In 1PA processes, the amount of energy from the laser that may be absorbed in a material varies linearly with the intensity of the laser. Specifically, photons incident on the material are absorbed by chromophores in the material, and the amount of absorption may be described by the Beer-Lambert Law, according to which absorption is a product of the chromophore's molar absorption coefficient, the concentration of the chromophore in the material, and the path length of the laser through the material. In 1PA processes, the laser beam may be provided continuously or as pulses.

A 2PA process is one that occurs when two photons of a single wavelength simultaneously strike a single chromophore molecule that absorbs half of the wavelength. That is, the two photons strike the chromophore with the correct amount of energy to be absorbed by the chromophore. The amount of 2PA that may occur in a material is a non-linear process that is proportional to the square of the incident intensity. As such, to accomplish 2PA absorption, the incident light should be tightly focused and provided as ultrashort pulses, e.g., with a femtosecond laser.

1PA and 2PA procedures have relative advantages and disadvantages to each other. For example, in 2PA processes, care must be taken to provide ultrashort pulses of light that are tightly focused, whereas in 1PA processes, the light may be provided continuously and not as tightly focused. Further, the light in 2PA processes should be more intense than in 1PA processes. Correspondingly, much of the light in 2PA processes is not absorbed such that it may be transmitted through the material, whereas, in 1PA processes, incident light need not be transmitted through the material according to the Beer-Lambert Law. Relatedly, in 2PA, when photons of a single wavelength strike the chromophore that absorbs half of that wavelength, the chromophore may fluoresce, which, in some applications, may be considered wasted energy. In 1PA, fluorescence may be minimized or avoided entirely. One conventionally accepted advantage of 2PA over 1PA is that 2PA absorption allows for more precise energy absorption at discrete locations inside the material than in 1PA procedures, where energy absorption may occur entirely along the path that the laser takes through the material.

RxSight® of Aliso Viejo, California, has described an intraocular lens in U.S. Pat. No. 10,010,406, that may be customized after cataract surgery. The lens comprises a so-called “modifying composition” that may be polymerized by irradiating the lens. This polymerization results in changing the radius of curvature of the lens. After the appropriate adjustment is achieved, a so-called “lock-in” procedure is performed to prevent further change to the lens's power.

### SUMMARY OF THE DISCLOSURE

A lens comprising an optic body may include a first portion comprised of a first material having a first spectral transmission profile and a second portion comprised of a second material having a second spectral transmission profile. A beam of light may be transmitted through the first portion to be absorbed by the second portion to effect a refractive-index change in the second material. The beam of light may have a wavelength greater than 380 nanometers, e.g., between 380 nanometers and 420 nanometers, or between 800 nanometers and 1000 nanometers. As such, the first material and the second material may comprise chromophores that absorb appropriate wavelengths of light based on the foregoing, or to absorb ultraviolet light that is harmful to the eye.

The lens may be irradiated at different spots with a beam of light or laser in order to effect a change to the index of refraction of the second material, and thus the lens overall. The properties, e.g., power density, of the beam may be changed over time and based on the spot being irradiated to achieve the desired refractive index change.

### BRIEF DESCRIPTION OF THE DRAWINGS

While the specification concludes with claims, which particularly point out and distinctly claim the subject matter described herein, it is believed the subject matter will be better understood from the following description of certain examples taken in conjunction with the accompanying drawings, in which like reference numerals identify the same elements and in which:

FIG. 1 depicts a schematic of an intraocular lens;

FIG. 2 depicts a cross section of an optic body of the intraocular lens of FIG. 1;

FIG. 3 reflects a plot of spectral transmission profiles for materials of the intraocular lens of FIG. 1

FIG. 4 reflects another plot of spectral transmission profiles for materials of the intraocular lens of FIG. 1

FIG. 5 depicts a flow chart setting forth a method for modifying a refractive index of the optic body of FIG. 2; and

FIG. 6 depicts a flow chart setting forth a method for locating a portion of the optic body of FIG. 2.

### MODES OF CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. For example, “about” or “approximately” may refer to the range of values±10% of the recited value, e.g. “about 90%” may refer to the range of values from 81% to 99%. In addition, as used herein, the terms “patient,” “host,” “user,” and “subject” refer to any human or animal subject and are not intended to limit the systems or methods to human use, although use of the subject invention in a human patient represents a preferred embodiment.

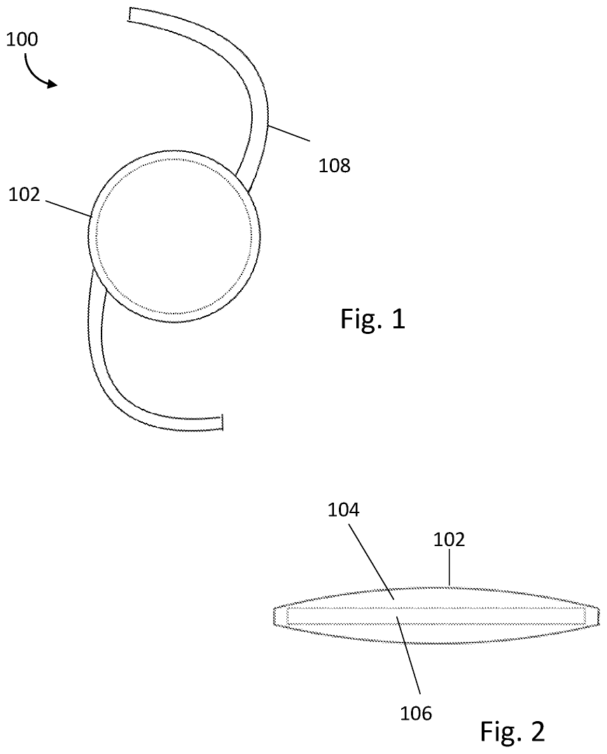

The disclosed subject matter is directed to a lens that may have its refractive index changed. Radiation, particularly from lasers, may be used to modify hydrophilic properties, and thus, the refractive index, of the lens. FIG. 1 reflects a top view of lens 100. As shown, lens 100 is an intraocular lens (“IOL”), however most of the subject matter described herein is applicable to other types of lenses, e.g., spectacle lenses and contact lenses. Therefore, the term lens as used herein is not limited to an IOL, but should include other types of lenses as well, including spectacle lenses and contact lenses.

Lens 100 comprises an optic body 102, a cross-section of which is reflected in FIG. 2. Where lens 100 is an IOL, lens 100 may also include haptics 108. Optic body 102 comprises a first portion or component 104 that abuts a second portion or component 106. Second portion 106 may be disposed or encapsulated in first portion 104, as shown in FIG. 1. Alternatively, second portion 104 may be disposed to only one side of first portion 102.

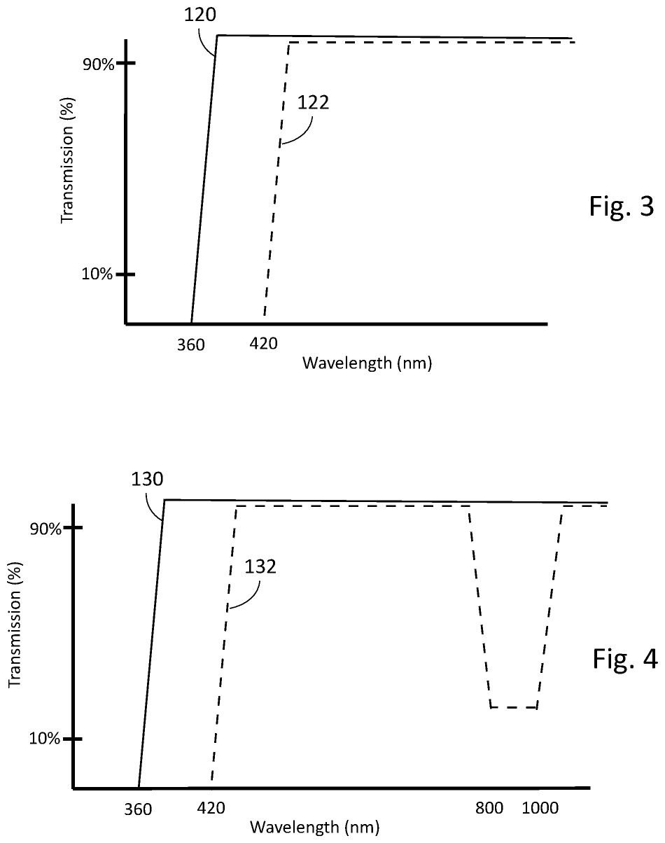

First portion 104 may be fabricated from a first material having a first transmission profile, i.e., first spectral transmission profile, and second portion 106 may be fabricated from a second material having a second transmission profile, i.e., second spectral transmission profile. The first transmission profile and second transmission profile may be selected such that the second material is capable of absorbing light of a wavelength or a range of wavelengths that is not absorbed by the first material. As a first example, the first material may be selected such that it absorbs light having wavelengths less than about 360 nanometers and transmits light having a range of wavelengths between about 380 nanometers and about 420 nanometers or more, while the second material is selected such that it absorbs at least a wavelength or sub-range of wavelengths in this range of wavelengths. With reference to FIG. 3, transmission profiles are provided in which transmission percentages are reflected as a function of wavelength. The first transmission profile of the first material is indicated with solid line and reference numeral 120, and the second transmission profile of the second material is indicated with dashed line and reference numeral 122. As such, a specific wavelength in the aforementioned range between about 380 and about 420 nanometers, e.g., about 390 nanometers, may pass through the first material to be absorbed in the second material.

As a second example, the first material may be selected such that it absorbs light having wavelengths less than about 400 nanometers (e.g., less than 360 nanometers) and transmits light having a range of wavelengths between about 800 nanometers or less and about 1000 nanometers or more while the second material is selected such that it absorbs at least a wavelength or sub-range of wavelengths in this range of wavelengths. With reference to FIG. 4, the first transmission profile of the first material is indicated with solid line and reference numeral 130, and the second transmission profile of the second material is indicated by a dashed line and reference numeral 132. As such, a specific wavelength in the aforementioned range between about 800 nanometers or less and about 1000 nanometers or more, e.g., about 900 nanometers, may pass through the first material to be absorbed in the second material.

As a third example, the first material may be selected such that it absorbs light having wavelengths less than a first wavelength and transmits light having a first range of wavelengths, while the second material is selected such that it absorbs at least some light having a second range of wavelengths that are about one and a half times greater to about five times greater than at least some of the wavelengths in the first range of wavelengths.

In greater detail, the first material may have a first spectral transmission profile characterized by a first cutoff wavelength and the second material may have a second spectral transmission profile characterized by a second cutoff wavelength that is greater than the first cutoff wavelength. Accordingly, wavelengths of incident light greater than the first cutoff wavelength may be readily transmitted through first portion 104, such that at a range of wavelengths of the light that reaches second portion 106 will be absorbed therein. More specifically, the term “cutoff wavelength” indicates that a material may, i.e., has a property of being able to, transmit therethrough a transmission percentage (such as between about 5% and 30%, e.g., 10%) of a wavelength of incident light that is equal or about equal to the cutoff wavelength. The term “cutoff wavelength” further indicates that the material may transmit therethrough less than or equal to the transmission percentage for a lesser wavelength of incident light in a first range of wavelengths that are less than the cutoff wavelength. The term “cutoff wavelength” further indicates that the material may transmit therethrough more than the transmission percentage for a greater wavelength of incident light in a second range of wavelengths that are greater than the cutoff wavelength. The transmission percentage of incident light that may pass through a material depends on the intensity of the incident light at an interface with the material and the material's thickness, as governed by the Beer-Lambert Law.

Preferably, the first cutoff wavelength and the second cutoff wavelength do not fall within the visible spectrum, although in some embodiments, they may fall on the edges of the visible spectrum. Such is preferred because first portion 104 and second portion 106 should be transparent to the visible spectrum of light, as is typical of most prescription lenses intended for daily or continuous use by a subject. Additionally, at least the first cutoff wavelength may be chosen to prevent harmful ultraviolet light from entering the eye. As such, the first cutoff wavelength may equal between about 350 nanometers and about 380 nanometers, e.g., about 365 nanometers, and the first spectral transmission profile may be further characterized by transmission of less than about 10% of incident light comprising a range of wavelengths that are less than the first cutoff wavelength. The second cutoff wavelength may also be in the ultra-violet spectrum or at least partially so, however it should be greater than the first cutoff wavelength. As such, the second cutoff wavelength may equal between about 380 nanometers and about 420 nanometers e.g., about 410 nanometers, and the second spectral transmission profile may be further characterized by transmission of less than about 10% of incident light comprising a range of wavelengths that are less than the second cutoff wavelength.

Inasmuch as lens 100 is intended to optionally be irradiated by a laser beam after it has been implanted in an eye of a subject to change the refractive index of the lens, it may be further desirable to provide the second cutoff wavelength as being in the near-infrared spectrum or close to it. In that manner, the laser beam may be provided as also having a wavelength in the near-infrared spectrum, which allows for a safer treatment than if the laser beam were to be provided as having a wavelength in the ultra-violet spectrum. Thus, the second cutoff wavelength may be in the near infrared spectrum (780 nanometers to 2500 nanometers) or near to it such that it may equal between about 700 nanometers and about 1300 nanometers, e.g., between about 800 nanometers and about 1000 nanometers, and the second spectral transmission may be further characterized by transmission of between about 5% and 30% of incident light comprising a range of wavelengths in the range of these second cutoff wavelengths. This increased transmission, as compared to the about 10%-or-less transmission described in the preceding paragraph, may further assist in measurements of lens 100's optical power following a modification to its refractive index, e.g., as described in method 200, below.

The spectral profiles may be achieved by providing a first chromophore in the first material or first portion 104 and a second chromophore in the second material or second portion 106. Preferably, the first chromophore is distributed evenly through the first material and the second chromophore is distributed evenly through the second material. The first chromophore may comprise benzophenone. Where the second cutoff wavelength is between about 380 nanometers and about 420 nanometers, the second chromophore may comprise benzotriazole. Where the second cutoff wavelength is between about 700 nanometers and about 1300 nanometers, the second chromophore may comprise a functional phthalocyanine (e.g., naphthalocyanine). Additionally, where the second cutoff wavelength is between about 700 nanometers and about 1300 nanometers, benzophenone or benzotriazole may also be included in the second material in addition to the functional phthalocyanine to further assist in preventing ultraviolet light from being transmitted through lens 100.

The first chromophore may be provided in the first material at a first concentration and the second chromophore may be provided in the second material at a second concentration. The first concentration may be between about 0.1 wt/wt % and about 10 wt/wt %, e.g., about 2 wt/wt %. The second concentration may also be between about 0.1 wt/wt % and about 10 wt/wt %, e.g., about 2 wt/wt %. At least in those instances where the first cutoff wavelength and the second cutoff wavelength are in the ultraviolet range or close thereto (e.g., less than 420 nanometers), it is possible to use the same chromophore. However, in such instances, the second chromophore may be provided in second portion 106 as having a greater concentration than the first chromophore in first portion 104. For example, the second chromophore may be provided at double the concentration of the first chromophore.

The first material and the second material may comprise, at least in part, a material having hydrophilic properties, or any other material that may be considered hydrophobic that nonetheless has some capacity to maintain water therein. Exemplary materials include: an acrylic material, polymethyl methacrylate, silicone, collagen, 2-hydroxyethyl methacrylate, poly(2-hydroxyethyl methacrylate), polyurethane, Collamer®, polycarbonate, allyl diglycol carbonate, and Trivex®. By modifying the hydrophilicity of these materials, e.g., by irradiating them using the techniques discussed below, the refractive index of a lens comprising one or more of these materials may be changed because water has a lower refractive index than these materials. Preferably, the first material and second material, when used in first portion 104 and second portion 106, may both have an equilibrium water content of between about 1% and about 50%, such as about 40% or between about 2% and 30% (e.g., about 4% or about 25%)

First portion 104 may be provided as having the form of a disc, which may have a varying thickness as shown in FIG. 2. Alternatively, it may have a uniform thickness. The maximum thickness of the disc of first portion 104 may be between about 0.5 millimeters and 4 millimeters, e.g., 1.5 millimeters. Second portion 106 may also have the form of a disc, which may have a uniform thickness as shown in FIG. 2. Alternatively, it may have a varying thickness. The maximum thickness of the disc of second portion 106 may be between about 0.1 millimeters and 2 millimeters, e.g., between about 0.3 millimeters and about 0.5 millimeters. These thicknesses are particularly applicable when lens 100 is an IOL, e.g., a monofocal intraocular lens, a toric intraocular lens, a multifocal intraocular lens, an extended depth of focus intraocular lens, an accommodating intraocular lens, a multi-component exchangeable intraocular lens, a pseudophakic intraocular lens, a piggyback intraocular lens, or a phakic intraocular lens.

By virtue of the embodiments illustrated and described herein, Applicant has devised a method and variations thereof for modifying the refractive index of a lens, such as lens 100. These methods may be performed while the lens is outside of a subject, or, in the case of an IOL, after the lens has been implanted in the subject, i.e., while the lens is disposed in the subject's eye. In those variation where the lens is irradiated while it is disposed in a subject's eye, and those embodiments where second portion 106 is not encapsulated in first portion 104, the lens may be implanted with second portion 106 disposed posteriorly to first portion 104, or at least a substantial portion thereof. Unlike the lens described in U.S. Pat. No. 10,010,406, lens 100 does not require a lock-in procedure to stabilize the lens's power. It is envisioned that lens 100 may have its refractive index modified many times after it has been implanted and the subject's eye has healed.

Techniques for using beams of light to irradiate spots of a material, including lenses, are known in the art. One accepted technique involves aligning a target material at the focus of a laser beam. The laser beam and lens are moved relative to each other such that the beam traces a path, e.g., a spiral trajectory or raster pattern, on the target. The intensity of the beam may be changed as the beam moves about the target, such that different and precise locations, i.e., spots, may be irradiated to receive differing power densities, i.e., energy per time per area or energy per time per volume.

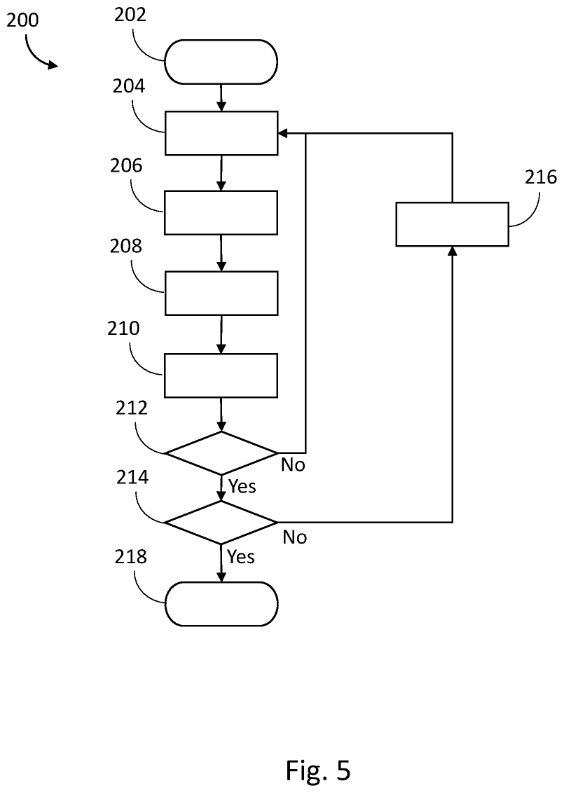

The flow chart of FIG. 5 describes a method 200 for modifying the refractive index of a lens, such as lens 100. In general, the lens is irradiated with a beam of light, such as a laser beam, having a wavelength that is greater than the first cutoff wavelength, e.g., about 360 nanometers. As such, where second portion 106 has a second cutoff wavelength of between about 380 nanometers and 420 nanometers, the beam of light may have a wavelength greater than the second cutoff wavelength, e.g., a wavelength of at least about 380 nanometers, such as between about 380 nanometers and about 410 nanometers, e.g., about 395 nanometers. Where second portion 106 has a second cutoff wavelength of between about 700 nanometers and about 1300 nanometers, the beam of light may have a wavelength greater than the second cutoff wavelength, e.g., a wavelength of at least about 700 nanometers or about 720 nanometers, such as between about 800 nanometers and about 1000 nanometers, e.g., about 900 nanometers. The beam should also have a numerical aperture between about 0.05 and 0.15, preferably about 0.1, such that spots of sufficiently small size in the lens, particularly in second portion 106, may be irradiated by the laser beam.

Method 200 is specifically described here with reference to lens 100. Method 200 begins at step 202. At step 202, the beam is activated and is initially directed to a first spot in second material 106. In those variations where the beam traces a spiral of spots on the lens, the first spot may be disposed proximate to a periphery of the lens such that the spiral trajectory of the laser moves inward toward the center of the lens. At step 204, the first spot is irradiated by the beam, which commences a first pass of the beam about the lens. Preferably, the beam is provided at a sufficient intensity and sufficient duration to irradiate the first spot with a power density between about 0 watts and about 0.5 watts per micrometer squared.

At step 206, the laser is moved along the spiral trajectory to a subsequent spot, e.g., a second spot, a third spot, a fourth spot, etc. At step 208, the subsequent spot is irradiated with the beam of light, which may be referred to herein as a “second beam of light” (or the “third beam of light,” or the “fourth beam of light”) at least because the beam of light irradiates the subsequent spots with differing properties, e.g., position of the beam's focus, and power density. It should be understood, however, that the subsequent beams of light are typically provided by the same source/laser as the beam of light provided in steps 202 and 204. Preferably, the subsequent beam is provided at a sufficient intensity and sufficient duration to irradiate the subsequent spot with a power density of between about 0 watts and about 0.5 watts per micrometer squared. In some variations of the method, the laser remains activated while the laser moves to the subsequent spot. In other variations, the laser may be blocked or shuttered while it is moved to the subsequent spot. Furthermore, the power density may be varied along the spiral trajectory. For example, the amplitude of the beam may be modified as it is moved about the spiral trajectory in order to vary the power density provided to each spot along the spiral trajectory.

At step 210, steps 206 and 208 are repeated, with additional beams of light until the full spiral trajectory has been traced, thereby completing the first pass of the beam about the lens.

At step 212, a determination is made as to whether a desired or predetermined total power density has been delivered to each spot. If not, the beam may be returned to the first spot such that additional passes of the laser about the lens may be performed by repeating steps 202-210 until the desired or predetermined total power density has been delivered to each spot. One reason why multiple passes of the laser may be desirable over a single pass is that heat damage to the lens may be avoided by irradiating the spots with smaller doses of energy, e.g., less than about 0.1 watts per micrometer squared. Depending on the refractive-index change to the lens that is desired, some spots receive a different cumulative total power density than other spots. Of course, damage to second portion 106 from over irradiation should be avoided. Therefore, it may be preferable to avoid providing a total power density of 0.6 watts per micrometer squared to any given spot.

The cumulative total power density that is to be provided to each spot in each pass or set of passes may be determined by various techniques. For example, wavefront aberrometry may be used. In such a procedure, light may be passed through the lens to output a wavefront. The wavefront may be analyzed and compared to a target wavefront, i.e., a wavefront that provides optical characteristics that would improve a subject's vision. A set of parameters, including, e.g., beam intensity, exposure time at each spot, and numerical aperture, may be determined from this comparison such that, when applied, the refractive index of the lens may be changed in a manner intended to achieve the target wavefront. As such, in some variations, a first set of parameters for the first pass may be the same as a second set of parameters for the second pass, although, as noted above, the parameters for each spot may differ within a given pass. Alternatively, it may be desirable to provide greater power densities in earlier passes to quickly get close to the desired power density and then to provide lesser power densities in subsequent passes to accurately achieve the desired power density.

Upon determining that the desired or predetermined total power density has been provided to each spot at step 212, the method continues to step 214, at which the lens that is analyzed to verify if the desired refractive-index change has been achieved. For example, an output wavefront from the modified lens may be compared to the target wavefront. If a difference remains, the refractive index may be fine-tuned by repeating steps 202-212. However, based on the new output wavefront, a new set of parameters (e.g., beam intensity, exposure time at each spot, and numerical aperture) may be determined for this next set of passes at step 216. Due to factors such as measurement error and a need for care, it may be preferable to achieve about 90-95% of the refractive change during a first set of passes comprising one or more passes, and then the remainder of the refractive change over one, two, or more subsequent sets of passes, each comprising one or more passes. At step 218, the desired refractive-index change has been achieved.

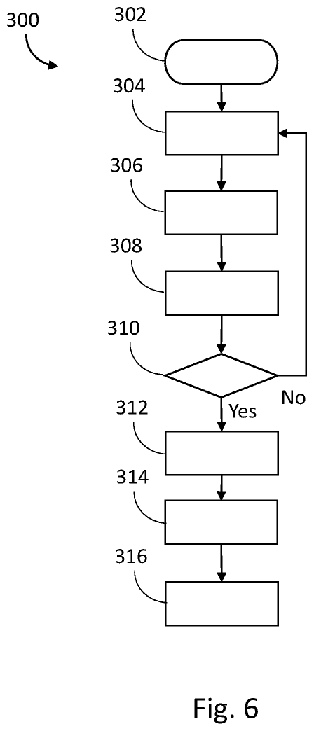

FIG. 6 sets forth another method, method 300, and variations that may be used to identify the position and orientation of a lens, such as lens 100, and particularly second portion 106. Method 300 may be used when lens 100 is disposed in an eye of a subject. This method uses a two-photon absorption technique with sufficiently low intensity that causes second portion 106 to fluoresce but that does not cause any changes to the hydrophilic properties of second portion of 106, and thus also does not cause any refractive-index changes thereto. Thus, the wavelength of the beam should be less than double the cutoff wavelength of the second portion 106 such that that the beam may readily pass through first portion 104

Method 300 begins at step 302, where an interrogation region comprising the lens may be determined. For example, the interrogation region may have a cylindrical profile with a diameter that is less than the diameter of the patient's pupil and a length that extends posteriorly about 8 millimeters from the plane of the subject's iris. At step 304, a laser beam may be focused at a spot in the interrogation region.

At step 306, which at least partially overlaps with step 304, fluorescence form the lens is monitored and measured, e.g., with a known computerized imaging apparatus that includes a photosensor capable of detecting fluorescence, a processor, and a non-transitory storage medium. If the focus of the laser beam falls in second portion 106, the lens will fluoresce. If the focus of the laser beam does not fall in the second portion 106, the lens will not fluoresce.

At step 308, a signal from the photosensor corresponding to detected fluorescence or a lack thereof may be transmitted to the processor, which associates the measurement with the spot's location in the interrogation region to create a fluorescence-measurement data point. The fluorescence measurement data point may then be stored in the storage medium.

At step 310, a determination is made as to whether the laser beam has been focused at all spots in the interrogation region (step 304). If not, then steps 304-308 are repeated until the laser beam has been focused at all spots at different locations in the interrogation region. For example, the laser-beam focus may be moved through the interrogation region along a spiral trajectory. After such has been completed, at step 312, a fluorescence-measurement data profile may be compiled comprising the various fluorescence-measurement data points.

At step 314, the fluorescence-measurement data profile may be analyzed to determine the position and orientation of the lens, particularly second portion 106. Specifically, the boundaries between non-fluorescing regions and fluorescing regions indicate surfaces of second portion 106. As such, both the position and the orientation of lens 100 may be determined. Accordingly, method 300 may be performed before method 200 to determine the position of second portion 106, and to thus focus the laser at spots located in second portion 106 when conducting method 200. Thus, in variations of method 300, a step 316 may include performing method 200 or any of its variations.

Any of the examples or embodiments described herein may include various other features in addition to or in lieu of those described above. The teachings, expressions, embodiments, examples, etc., described herein should not be viewed in isolation relative to each other. Various suitable ways in which the teachings herein may be combined should be clear to those skilled in the art in view of the teachings herein.

Having shown and described exemplary embodiments of the subject matter contained herein, further adaptations of the methods and systems described herein may be accomplished by appropriate modifications without departing from the scope of the claims. In addition, where methods and steps described above indicate certain events occurring in certain order, it is intended that certain steps do not have to be performed in the order described but in any order as long as the steps allow the embodiments to function for their intended purposes. Therefore, to the extent there are variations of the invention, which are within the spirit of the disclosure or equivalent to the inventions found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

1-117. (canceled)

118. A lens comprising:
an optic body, including:
a first portion comprised of a first material having a first spectral transmission profile characterized by a first cutoff wavelength; and
a second portion comprised of a second material having a second spectral transmission profile characterized by a second cutoff wavelength that is greater than the first cutoff wavelength.

119. The lens of claim 118, in which the first portion abuts the second portion.

120. The lens of claim 119, in which the second portion is disposed in the first portion.

121. The lens of claim 119, in which the first cutoff wavelength equals a wavelength that is between about 350 nanometers and about 380 nanometers.

122. The lens of claim 121, in which the second cutoff wavelength equals a wavelength that is between about 380 nanometers and about 420 nanometers.

123. The lens of claim 121, in which the second cutoff wavelength equals a wavelength that is between about 700 nanometers and about 1300 nanometers.

124. The lens of claim 121, in which the first material and the second material are transparent across the visible spectrum.

125. The lens of claim 121, in which the first material and the second material both have an equilibrium water content of between about 1% and about 50%.

126. The lens of claim 121, in which the first portion comprises a first disc and the second portion comprises a second disc.

127. A method of adjusting a refractive index of a lens, comprising:
providing the lens, the lens comprising an optic body, including:
a first portion comprised of a first material having a first spectral transmission profile characterized by a first cutoff wavelength, and
a second portion comprised of a second material having a second spectral transmission profile characterized by a second cutoff wavelength that is greater than the first cutoff wavelength; and
irradiating the lens with a beam of light having a beam wavelength that is less than the second cutoff wavelength such that the first material transmits the beam of light and second material absorbs the beam of light.

128. The method of claim 127, in which the first cutoff wavelength and the second cutoff wavelength are not in the visible spectrum.

129. The method of claim 128, in which the first cutoff wavelength and the second cutoff wavelength are in the ultraviolet spectrum.

130. The method of claim 129, in which the beam wavelength equals between about 380 nanometers and about 410 nanometers.

131. The method of claim 130, in which the beam wavelength equals about 395 nanometers.

132. The method of claim 128, in which the first cutoff wavelength is in the ultraviolet spectrum and the second cutoff wavelength is in the near-infrared spectrum.

133. The method of claim 132, in which the beam wavelength equals between about 800 nanometers and about 1000 nanometers.

134. The method of claim 133, in which the beam wavelength equals about 900 nanometers.

135. The method of claim 132, in which the operation of irradiating the lens is performed while the lens is disposed in an eye, with the second portion disposed posteriorly to at least some of the first portion.

136. The method of claim 127, further comprising:
determining an interrogation region comprising the lens;
focusing an interrogation laser beam at a first location in the interrogation region;
during the operation of focusing the interrogation laser beam at the first location in the interrogation region, measuring fluorescence from the lens to obtain a fluorescence measurement;
repeating the steps of focusing the interrogation laser beam and measuring fluorescence to create a fluorescence-measurement data profile; and
based on the fluorescence-measurement data profile, determining the location of the second portion of the lens.

137. The method of claim 136, in which the operation of determining the location of the second portion further comprises determining an orientation of the second portion.
